(19) United States
(12) Patent Application Publication
(10) Pub. No.: US 2019/0055368 A1
(43) Pub. Date: Feb. 21, 2019

(54) Method of Preparing Single-Phase Modified Sodium Hyaluronate Gel

(71) Applicant: Hangzhou Singclean Medical Products Co., Ltd., Hangzhou (CN)

(72) Inventors: Xijiang Feng, Hangzhou (CN); Yan Chen, Hangzhou (CN); Weiqing Sun, Hangzhou (CN)

(21) Appl. No.: 16/022,788

(22) Filed: Jun. 29, 2018

Related U.S. Application Data
(63) Continuation of application No. PCT/CN2017/111286, filed on Nov. 16, 2017.

(30) Foreign Application Priority Data
Aug. 16, 2017 (CN) 201710699814.2

(51) Int. Cl. C08J 3/075; A61L 27/52; A61L 27/20; C08J 3/24

(52) U.S. Cl. CPC C08J 3/075; A61L 27/52; A61L 27/20; C08J 2305/08; A61L 2400/06; A61L 2430/34; C08J 3/24

(57) ABSTRACT

A method of preparing a single-phase modified sodium hyaluronate gel, comprising preparing a sodium hyaluronate solution with a mass fraction between 5% to 15% in an alkaline condition at a pH value between 11 to 14, wherein the sodium hyaluronate has a molecular weight between 1.5 million to 4 million Daltons; adding a cross-linking agent to a solution of step (1), wherein the cross-linking agent and the sodium hyaluronate has a molar ratio between 9% to 15%; rapidly mixing for 20 to 40 minutes to form a gel; allowing to stand after subjecting to a water bath at constant temperature; dialyzing with a dialysis membrane to remove un-reacted cross-linking agent and hydroxide ion; homogenizing; and adding a mobile phase and mixing sufficiently to obtain a high viscosity stabilized single-phase modified sodium hyaluronate gel.

### CROSS REFERENCE TO RELATED APPLICATIONS

This application is a continuation application of PCT Application No. PCT/CN2017/111286 filed on Nov. 16, 2017, which claims priority to Chinese Patent Application No. 201710699814.2 filed on Aug. 16, 2017, the entire contents of which are incorporated herein by reference.

### FIELD OF TECHNOLOGY

The present application relates to a method of preparing a single-phase modified sodium hyaluronate gel, and the product produced by the method. The product is suitable for use as the filling and repair of moderate and severe facial wrinkles or folds. The present application belongs to the field of implantable medical cosmetology.

### BACKGROUND TECHNOLOGY

In medical cosmetology, medical means including equipment, drugs, instruments and surgery are used in order to achieve a series of treatments for the purposes of changing the external appearance and color of the human body, partially improving the physiological functions thereof, and enhancing the aesthetics of the human body. Minimally invasive injection and implant for plastic surgery have developed rapidly, which exert the minimal damage to normal tissue, the minimal inflammatory response, the minimal swelling and blood stasis, the minimal complications, and the minimal scarring. Moreover, the treatment time is short, the patient suffers from minimal pain, the postoperative recovery is quick, the treatment effect is good, the safety is high, the patient does not need to be hospitalized, etc. The healthy and natural properties of hyaluronic acid are particularly prominent among the injection category. Since the cost of the injection is relatively low, the operation time is relatively short, the injection process will not cause significant pain and the body function will not be affected; recovery time is not necessary and it is easily accepted by the consumers. Its popularity is gradually increasing and injection of hyaluronic acid for cosmetic purpose is becoming the trend.

Hyaluronic acid (abbreviated as HA) was first isolated from the vitreous body of bovine eye. It is widely present in the extracellular matrix of the human connective tissue. Hayluronic acid is a linear polysaccharide composed of repeated units of disaccharide. The disaccharide unit is composed of D-glucuronic acid and N-acetyl-glucosamine, linked via alternating β 1-3 bonds and β 1-4 bonds. Hyaluronic acid has no species and tissue specificity, has good histocompatibility, and the body rarely triggers an immune response against hyaluronic acid. Hyaluronic acid has high hydrophilicity. Such a physicochemical property allows hyaluronic acid to remain in a gel state even at a very low concentration. Hyaluronic acid increases in volume after absorbing water, and generates swelling pressure against the surrounding such that it may support the tissues around it. However, the half-life of natural hyaluronic acid in tissue is only 1 to 2 days, and it will be degraded into CO₂ and H₂O by hyaluronidase or oxygen free radicals in the liver. Hyaluronic acid also has the characteristic of isovolumic degradation, i.e., when a portion of the hyaluronic acid is degraded, the remaining molecules can absorb more water content to maintain the total volume until all the molecules are completely degraded. Hyaluronic acid content decreases with age, which directly leads to the loss of water content in the skin thereby forming wrinkles. Therefore, hyaluronic acid is clinically applied in skin rejuvenation treatment such as improving wrinkles and increasing tissue capacity. Thus, in order for hyaluronic acid to become an ideal skin filling material, it must be modified and cross-linked. In this way, a product having a more stable molecular structure and lasting for longer can be obtained. Among different species or tissues, hyaluronic acid has no antigen-specificity and therefore it rarely causes allergic reactions. Meanwhile, hyaluronic acid has good conformational rigidity and extremely strong water-restraining function. Due to its excellent biocompatibility and filling effect, it is gradually replacing collagen and becoming the mainstream skin filling material at the present time.

On the current market, the more common products are the hyaluronic acids which are sufficiently modified using a sufficient amount of cross-linking agent, and artificially sieved to obtain solid granular gel block with much higher rigidity and strength than the native hyaluronic acid, thereby forming a bi-phase gel. Due to the difference in body conditions, syndromes such as redness, swelling, pain, inflammation, foreign body sensation, and delayed allergy are frequently seen clinically.

### SUMMARY

The objective of the present application is to provide a method of preparing a single-phase sodium hyaluronate gel. The product prepared by the method has good biocompatibility and excellent resistance to enzymatic degradation. Meanwhile, the method is easy to operate and can easily realize the production of industrialized medical device products. In order to achieve the above objective, the present application includes the following steps:

A method of preparing a single-phase modified sodium hyaluronate gel, including the following steps:
(1) preparing a sodium hyaluronate solution with a mass fraction between 5% to 15% in an alkaline condition at a pH value between 11 to 14, wherein the sodium hyaluronate has a molecular weight between 1.5 million to 4 million Daltons;
(2) adding a cross-linking agent to a solution of step (1), wherein the cross-linking agent and the sodium hyaluronate has a molar ratio between 9% to 15%; rapidly mixing for 20 to 40 minutes to form a gel;
(3) allowing the gel of step (2) to stand after subjecting the gel of step (2) to a water bath at constant temperature;
(4) dialyzing the gel of step (3) with a dialysis membrane to remove un-reacted cross-linking agent and hydroxide ion;
(5) homogenizing; and
(6) adding a mobile phase and mixing sufficiently to obtain a high viscosity stabilized single-phase modified sodium hyaluronate gel.

Preferably, the polymer is originated from natural source. Natural polymer has better biocompatibility. That is, the use of natural polymer will lead to a smaller risk of inflammatory reaction. In step (1), the sodium hyaluronate is a sodium hyaluronate produced by bacterial fermentation.

In a preferred embodiment, in step (1), the alkaline condition is selected from potassium hydroxide or sodium hydroxide, wherein the pH value is between 13 to 14.

In a preferred embodiment, in step (2), the cross-linking agent is selected from a compound of biological multi-functional molecule selected from the group consisting of epoxide, halohydrin and divinyl sulfone.

In a preferred embodiment, the epoxide is a compound selected from the group consisting of 1,4-butanediol diglycidyl ether (as known as 1,4-bis(2,3-epoxypropoxy)butane), 1-(2, 3-epoxypropyl) 2,3-epoxycyclohexane and 1,2-ethanediol diglycidyl ether.

In a preferred embodiment, in step (3), the constant temperature is between 27 to 60° C.

In a preferred embodiment, the constant temperature is between 30 to 50° C.

In a preferred embodiment, in step (4), the dialysis membrane has a dialysis molecular weight of 20,000 Daltons.

In a preferred embodiment, in step (4), the dialysis membrane has a dialysis molecular weight of 15,000 Daltons.

In a preferred embodiment, step (5) further comprising adding an anesthetic before the homogenizing.

In a preferred embodiment, the anesthetic is lidocaine hydrochloride; the anesthetic has a mass content between 0.1% to 0.5%.

In a preferred embodiment, the mass content is between 0.2% to 0.4%.

In a preferred embodiment, in step (6), the mobile phase is the same as a raw material of the sodium hyaluronate which has been cross-linked, and the mobile phase is prepared by bacterial fermentation; wherein the mobile phase and the raw material have a same molecular content; wherein the mobile phase and the gel has a consistent content; wherein the mobile phase has a total mass ratio of 5% to 50%.

In a preferred embodiment, the mobile phase has a total mass ratio of 10% to 30%.

In a preferred embodiment, the method includes dissolving the sodium hyaluronate with a molecular weight of 2 million Daltons in an alkaline solution at pH 14; adding 12% of the cross-linking agent, wherein the cross-linking agent is 1,4-butanediol diglycidyl ether; rapidly mixing for 30 minutes; cross-linking at 40° C. in the water bath; dialyzing with the dialysis membrane of 1.5 million Daltons; adding 0.2% to 0.4% of lidocaine hydrochloride; adding 20% of the mobile phase; thereby obtaining the single-phase modified sodium hyaluronate gel.

In a preferred embodiment, the single-phase sodium hyaluronate gel is sterilized by moist heat and then sealed and stored in a vial or pre-filled syringe.

The gel of the present application is preferably administered by injection.

### BRIEF DESCRIPTION OF THE DRAWINGS

FIG. 1 is a figure showing the results of a bi-phase granular gel prepared according to the existing method observed under the microscope.

FIG. 2 is a figure showing the results of a single-phase homogenous gel prepared according to the method of the present application observed under the microscope.

FIG. 3 is a graph comparing the curves of enzymatic degradation between Example 1, Example 3, and the bi-phase gel control.

### DETAILED DESCRIPTION

The present application provides a biocompatible single-phase cross-linked gel, which effectively avoids the disadvantages of the bi-phase gel described in the background. Meanwhile, the biocompatible single-phase cross-linked gel has the advantages of being easy to use and having longer duration in clinical application.

Starting from researching the degree of modification of hyaluronic acid in the human body itself, the present application has adopted a non-saturated cross-linking method through the control of the preliminary parameters, and further perform homogenization, to obtain a single-phase, polymeric, high-viscosity, injectable and long-lasting biocompatible sodium hyaluronate gel. The biocompatibility and physicochemical properties of the product are greatly improved. Furthermore, in order to enhance the comfort level of experience in clinical use, anesthetic lidocaine hydrochloride is added during the production process. The entire process is easy to implement and the result is stable and reliable.

The single-phase modified sodium hyaluronate gel prepared according to the above method may fill, separate or replace a biological tissue, or increase the volume of the tissue, or supplement or replace a biological fluid. Preferably, the gel is used for filling, separating or replacing the biological tissue or increasing the volume of the tissue, e.g. as a material for therapeutic application, increasing the volume of the vocal cord, increasing the volume of the esophagus, urethral sphincter or other organs, etc., or for filling wrinkle, covering scar or enriching the lip for cosmetic purposes. Preferably, the gel constitutes a matrix including at least one dispersed active body. The gel is then used as a carrier for the active body gradually released from the injected liquid or biological tissue.

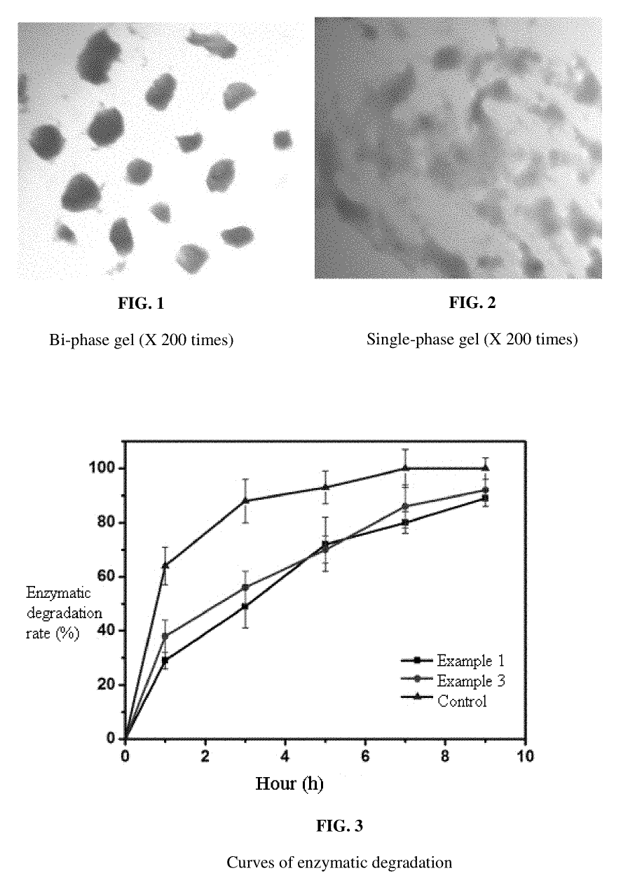

The single-phase gel is different from the conventional bi-phase gel. Observation of the microscopic structure shows that the conventional bi-phase gels are distinguished by the particle sizes as shown in FIG. 1, while the single-phase homogenized gel is shown in FIG. 2.

The bi-phase contains solid particles and a liquid phase. This leads to localized area which is not smooth during application. Thus, absorption and degradation of the bi-phase gel are different in the body. Meanwhile, the single-phase gel is a very stable colloidal phase, which is very similar to the degree of modification in hyaluronic acid present in the human body itself. The single-phase gel has a stable structure, a high affinity to the tissue, a more natural shaping effect, a significant lifting effect, is effective in improving the unevenness of the postoperative skin surface, and is soft and elastic. The high viscosity of the gel means that the gel has a strong tendency of restructuring, instead of spreading or separating. The excellent corresponding shear viscosity may effectively resist the shear force generated after the injection, thereby reducing diffusion and movement, resisting deformation caused by external force, and being more stable. Therefore, the high viscosity cohesiveness of the gel contributes to the high compatibility and long-term sustainability in vivo.

It should be specifically noted that the property of high viscosity of the single-phase gel of the present application does not solely mean that the gel has high absolute value of viscosity in specific test conditions, but it needs to be combined with its elastic properties for a comprehensive evaluation. In the industry, the phase angle α is a measure of the rheological properties of the gel. tg α is the ratio of viscosity to elasticity. The gel of the present application generally has a larger phase angle than the bi-phase gel, and the viscosity performance is more outstanding.

The single-phase sodium hyaluronate gel prepared according to the present application has outstanding performance, which further reduces the risk of inflammatory reaction and the appearance of granuloma. The prolonged retaining period in the body provides opportunities for longer-interval medical interventions, thereby improving the quality of life of the patients.

The modified sodium hyaluronate gel prepared according to the present application is more injectable in vivo than other gels having the same degree of cross-linking, and has longer persistence.

The following examples are given to illustrate the method and product according to the present application and to assist with the understanding of the present application by way of illustration only, but they are by no means intended to limit the scope of the present application.

### Example 1

2.02 g of sodium hyaluronate with a molecular weight of 1.60 million Daltons was weighed in a beaker and 15.05 g of 1% sodium hydroxide solution was added to fully dissolve the sodium hyaluronate. The cross-linking step was carried out in an alkaline medium and very strong ether bonds were easily formed. 60 μl of divinyl sulfone was added, thoroughly mixed, and allowed to react for 4 hours at 50° C. and stand overnight to obtain the cross-linked gel.

### Example 2

The gel of Example 1 was placed in a dialysis membrane bag with a dialysis molecular weight of 15,000 Daltons for dialysis, in order to remove un-reacted cross-linking agents and excess hydroxide ions. 0.3% lidocaine hydrochloride, which was filtered by 0.2 μm microfiltration membrane, was then added to adjust the pH to neutral, and then homogenized. 10.06 g of mobile phase was added and thoroughly mixed to obtain a homogenous single-phase gel.

The gel was packaged in pre-filled syringe and subjected to moist heat sterilization at 121° C. for 30 minutes.

The product was observed under a Winner 99D Particle Image Analyzer, and the results are shown in FIG. 2.

### Example 3

5.03 g of sodium hyaluronate with a molecular weight of 2.3 million Daltons was weighed in a beaker and 55.2 g of a 1% sodium hydroxide solution was added to fully dissolve the sodium hyaluronate. 321 μl of 1,4-butanediol diglycidyl ether was added, thoroughly mixed, and then allowed to react for 4 hours at 40° C. and stand overnight to obtain the cross-linked gel. The gel was then placed in a dialysis membrane bag for dialysis, in order to remove un-reacted cross-linking agents and excess hydroxide ions. 0.3% of lidocaine hydrochloride, which was filtered by 0.2 μm microfiltration membrane, was added to adjust the pH to neutral, and then homogenized. 52.1 g of mobile phase was added and thoroughly mixed to obtain a homogenous single-phase gel.

### Example 4

10.02 g of sodium hyaluronate with a molecular weight of 1.9 million Daltons was weighed in a beaker and 160.1 g of a 1% sodium hydroxide solution was added to fully dissolve the sodium hyaluronate. 550 μl of 1,4-butanediol diglycidyl ether was added, thoroughly mixed, and then allowed to react for 4 hours at 40° C. and stand overnight to obtain the cross-linked gel. The gel was then placed in phosphate buffered saline for dialysis, in order to remove un-reacted cross-linking agents and excess hydroxide ions. 0.3% of lidocaine hydrochloride, which was filtered by 0.2 μm microfiltration membrane, was added to adjust the pH to neutral, and then homogenized. 105.0 g of mobile phase was added and thoroughly mixed to obtain a homogenous single-phase gel.

The gels of the above examples were tested for elasticity and viscosity using a rotational rheometer. The test method was dynamic frequency scanning at a test temperature of 25° C. and a frequency change range between 0.05 to 10 Hz. The values of elastic modulus (G′) and viscous modulus (G″) at 1 Hz were compared.

For each set of samples in the examples, 5 parallel samples were taken and installed in the injection needles. The syringe was pushed to remove the small amount of air from the front end until a gel droplet appeared at the needle tip. The syringe was placed on a tension machine and the plunger was pushed at a speed of 20 mm/min. The pressure was recorded and the average value was calculated.

A comparison between the test results of the single-phase gel according to the present application and a bi-phase gel commercially available on the current market is shown in the following table:

| | Single-phase | Single-phase | | |
|---|---|---|---|---|
| | Gel | Gel | Single-phase Gel | |
| | in Example 1 | in Example 3 | in Example 4 | Bi-phase Gel |
| Elasticity | 193 | 186 | 190 | 693 |
| (Pa, 1 Hz) | | | | |
| Viscosity | 50 | 48 | 55 | 130 |
| (Pa, 1 Hz) | | | | |
| Pushing | 10 | 9 | 10 | 19 |
| Force (N) | | | | |

The in vitro resistance to enzymatic degradation of the gel may indirectly reflect its lasting time in the human body. In the present application, the gels of Examples 1 and 3 and the commercially available bi-phase gel control were subjected to an enzymatic degradation test at a hyaluronidase concentration of 7 U/ml. The results are shown in the following table. The curves of enzymatic degradation are shown in FIG. 3.

| | Enzymatic Degradation | | | | |
|---|---|---|---|---|---|
| | Rate (%) | | | | |
| Hour (h) | 1 | 3 | 5 | 7 | 9 |
| Example 1 | 29 | 49 | 72 | 80 | 89 |
| Example 3 | 38 | 56 | 70 | 86 | 92 |
| Control | 64 | 88 | 93 | 100 | 100 |

What is claimed is:

1. A method of preparing a single-phase modified sodium hyaluronate gel, comprising the following steps:
(1) preparing a sodium hyaluronate solution with a mass fraction between 5% to 15% in an alkaline condition at a pH value between 11 to 14, wherein the sodium hyaluronate has a molecular weight between 1.5 million to 4 million Daltons;
(2) adding a cross-linking agent to a solution of step (1), wherein the cross-linking agent and the sodium hyaluronate has a molar ratio between 9% to 15%; rapidly mixing for 20 to 40 minutes to form a gel;
(3) allowing the gel of step (2) to stand after subjecting the gel of step (2) to a water bath at constant temperature;
(4) dialyzing the gel of step (3) with a dialysis membrane to remove un-reacted cross-linking agent and hydroxide ion;
(5) homogenizing; and
(6) adding a mobile phase and mixing sufficiently to obtain a high viscosity stabilized single-phase modified sodium hyaluronate gel.

2. The method according to claim 1, wherein in step (1), the sodium hyaluronate is a sodium hyaluronate produced by bacterial fermentation.

3. The method according to claim 1, wherein in step (1), the alkaline condition is selected from potassium hydroxide or sodium hydroxide, wherein the pH value is between 13 to 14.

4. The method according to claim 1, wherein in step (2), the cross-linking agent is selected from epoxide, halohydrin or divinyl sulfone.

5. The method according to claim 4, wherein the epoxide is a compound selected from the group consisting of 1,4-butanediol diglycidyl ether, 1-(2, 3-epoxypropyl) 2,3-epoxycyclohexane and 1,2-ethanediol diglycidyl ether.

6. The method according to claim 1, wherein in step (3), the constant temperature is between 27 to 60° C.

7. The method according to claim 6, wherein the constant temperature is between 30 to 50° C.

8. The method according to claim 1, wherein in step (4), the dialysis membrane has a dialysis molecular weight of 20,000 Daltons.

9. The method according to claim 1, wherein in step (4), the dialysis membrane has a dialysis molecular weight of 15,000 Daltons.

10. The method according to claim 1, wherein step (5) further comprising adding an anesthetic before the homogenizing.

11. The method according to claim 10, wherein the anesthetic is lidocaine hydrochloride; the anesthetic has a mass content between 0.1% to 0.5%.

12. The method according to claim 11, wherein the mass content is between 0.2% to 0.4%.

13. The method according to claim 1, wherein in step (6), the mobile phase is the same as a raw material of the sodium hyaluronate which has been cross-linked, and the mobile phase is prepared by bacterial fermentation; wherein the mobile phase and the raw material have a same molecular weight; wherein the mobile phase and the gel has a consistent content; wherein the mobile phase has a total mass ratio of 5% to 50%.

14. The method according to claim 13, wherein the mobile phase has a total mass ratio of 10% to 30%.

15. The method according to claim 1, comprising dissolving the sodium hyaluronate with a molecular weight of 2 million Daltons in an alkaline solution at pH 14; adding 12% of the cross-linking agent, wherein the cross-linking agent is 1,4-butanediol diglycidyl ether; rapidly mixing for 30 minutes; cross-linking at 40° C. in the water bath; dialyzing with the dialysis membrane of 1.5 million Daltons; adding 0.2% to 0.4% of lidocaine hydrochloride; adding 20% of the mobile phase; thereby obtaining the single-phase modified sodium hyaluronate gel.

16. A method of filling, replacing or isolating a biological tissue, or increasing a volume of the biological tissue, or supplementing or replacing a biological material in medical cosmetology, comprising administering an effective amount of the single-phase modified sodium hyaluronate gel prepared by the method of claim 1 to a subject in need thereof.
